(19) United States
(12) Patent Application Publication
(10) Pub. No.: US 2009/0247835 A1
(43) Pub. Date: Oct. 1, 2009

(54) METHOD AND A DEVICE FOR ADAPTING EEG MEASUREMENT SIGNALS

(75) Inventor: Juha Voipio, Kirkkonummi (FI)

Correspondence Address: YOUNG & THOMPSON, 209 Madison Street, Suite 500, ALEXANDRIA, VA, 22314, US

(73) Assignee: BRAINSCOPE OY, KIRKKONUMMI (FI)

(21) Appl. No.: 12/280,164

(22) PCT Filed: Feb. 22, 2007
(86) PCT No.: PCT/FI2006/000062

§ 371 Date: Oct. 22, 2008

(51) Int. Cl. A61B 5/0476; A61B 5/0402

(52) U.S. Cl. 600/301; 600/544; 600/509

(57) ABSTRACT

A method and an apparatus (108) for adapting a received EEG measurement signal to the characteristic range of an ECG measurement signal according to a number of predetermined factors. The suggested solution enables utilization of an ordinary ECG measuring instrument (110) and related infrastructure also in EEG measurements.

### FIELD OF THE INVENTION

The present invention relates generally to biomedical engineering, and more exactly to EEG (electroencephalography) and ECG (electrocardiography, EKG) measuring instruments and technology.

### BACKGROUND OF THE INVENTION

Electrical activity of excitable cells, such as heart muscle cells or neurons within the brain, gives rise to electrical signals that can be detected on the skin. Two well-established techniques with a wide range of clinical applications are then electroencephalography (EEG) that measures activity of the brain, and the electrocardiography (ECG, EKG) that measures activity of the heart. In EEG, the measured voltage signals mainly arise from brain cortical synaptic currents, and reflect the level of excitation and degree of synchrony in brain neuronal network. While spreading from brain tissue to scalp, EEG signals get smeared and attenuated especially because of the low conductivity of the skull. Typical EEG signal amplitudes and frequencies that are monitored in clinical applications range from 5 to 250 RV and 0.5 to 80 Hz, respectively, with signals crucial for diagnostic purposes consisting mainly of frequencies between 2 to 30 Hz. In the heart, muscle cells are electrically coupled, and therefore all cells are recruited to a synchronous action potential that rapidly spreads through the heart during each contraction cycle. This activity generates the ECG signal that is typically measured with three or more electrodes positioned on the skin of the chest (or leg and arms). The ECG signal has a characteristic waveform with peak amplitudes up to approximately 5 mV depending on electrode positions, and a bandwidth with the main frequency content within 0.5 to 100 Hz.

ECG is a widely used diagnostic tool also available in emergency care in all developed countries. This implies that the ECG devices are part of standard equipment in ambulances, emergency rooms, intensive care units, health centers etc, and medical staff in such units is trained to carry out ECG measurements. BOG is commonly measured using a multi-channel ECG device on several electrode locations (chest, limbs) for diagnostic purposes. In long-term monitoring and emergency situations less channels and electrode locations are needed to give clinically acceptable findings. ECG electrodes, cables and connectors are typically coded using different colors, which may be different in different countries and continents (Europe, USA).

EEG measurements are often conducted for diagnostic purposes to study disorders of brain electrical activity of a target person (called hereinafter a “patient”). Such disorders include altered consciousness and neurological symptoms due to seizure disorders (e.g. epilepsy), inflammation and structural lesions of the brain tissue, disturbances of blood flow (stroke) and metabolic disorders (e.g. intoxication) of brain. Abnormal brain electrical activity is recognised from the EEG signal as abnormal constant or fluctuating variations in amplitude, frequency or shape of the BEG signal. Variations even in the normal BEG are considerable and influenced by the age, vigilance (wake-sleep), used medication, etc. In addition, several artefacts may disturb the signal, A diagnostic EEG study is performed using several measurement electrodes and locations (more than 20) with a multi-channel EEG device. The application of the electrodes to the scalp followed by difficult and complicated use of the EEG device not forgetting the interpretation of the findings requires specialized personnel. The EEG findings are used for the assessment of proper treatment, prognosis, state changes, and the results of the treatment.

As the EEG devices for multichannel (more than 20) recordings are expensive and difficult to use, they are not found on emergency wards. There are some commercial, smaller EEG monitoring devices with e.g. 2, 4, or 8 measurement channels constructed to be used in emergency room. Even these devices are high-priced (10.000-30.000 EUR), use expensive technology required in multichannel recording, analysis and display of EEG, and are therefore available only on a few specialized wards.

There are also multi-modal monitoring devices used on emergency wards. Typically this kind of device consists of a routine ECO monitor, respiratory, pulse wave, and non-invasive oxygen saturation measure. Some of the monitors also comprise one or two channels for EEG measurement. While the simple BCG monitors are used in primary care, these multi-modal monitoring devices are often the most versatile, expensive, and big in size, being therefore used on more specialized wards including operating and recovery room but not in primary care equipped with simple ECG monitoring devices.

Quite frequently in emergency care, brain disorders can be recognized even on the basis of few channels or just a single channel EEC recording. This is especially likely in most critical situations and in follow-up of drug therapy given to the patient, Similar EEG follow-up is also performed during anaesthesia in operating room using only one channel BEG. Such EEG monitoring is often possible by simply fixing the measuring electrodes to the forehead (frontal area) of the scull of the patient. The findings in one-channel BEG are less complicated to be interpreted. As the consequences of brain disorders may be serious or even life threatening without adequate therapy, there is a distinct need in emergency medicine for widely available BEG monitoring resources even with limited number of electrodes and features.

As mentioned, ECG monitoring devices are used and found routinely in almost all places treating acutely ill patients (emergency room, intensive care and ordinary wards, operating rooms, health centers, ambulance units, even in first aid rooms of meeting buildings, airplanes etc), All the personnel working in the primary emergency care are basically capable of performing an BCG recording. ECG measurements are transmitted electronically to specialists Thorn ambulances or distant health centers and there has been an extensive development of other aspects of the ECG infrastructure technology (storage, display, archiving etc). ECG measures and devices are in world wide routine use also in heal care systems in countries with few or no access to BEG monitoring facilities. Even in well-equipped general or university hospitals with neurological, emergency and intensive care units, ECG monitoring devices may outnumber EEG devices by dozens to one.

One could ponder whether rare EEG and better-established ECG monitoring devices contain substantial differences preventing direct cross-use thereof, while considering also the related infrastructure (measuring, display, storage, telemetry and telemedicine etc) and various properties of the corresponding measurement signals.

The recording bandwidths of ECG and EEG devices are quite similar, with the lower cut-off frequency being typically about 0.1 to 0.5 Hz and the higher cut-off at around 100 Hz. Therefore, the signals within the frequency band from 2 to 30 Hz, that is crucial in the EEG-based diagnostics of acute brain disorders, do not get distorted in any standard ECG devices. However, the existing BCG devices cannot be used for measuring BEG due to a much smaller peak-to-peak amplitude variation of EEG signals compared to ECG signals (typical amplitudes of 5-10 to 200-250 μV and 1 to 5 mV, respectively). Characteristics of the ECG signals have been naturally taken into account in the design of ECG devices thus affecting various component selections, signal-to-noise ratio, calibration, and signal visualization at the output (a display, a plotter, etc).

Publication U.S. Pat. No. 5,287,859 discloses an EEG arrangement to be used by general physicians in their private offices instead of fully separate, expensive EEG units. One embodiment of the arrangement partly utilizes a common multi-channel ECG apparatus the amplifiers of which are harnessed for amplifying already pre-amplified, analogue EEG electrode signals; thus e.g. the original leads and electrodes of the ECG device are not capitalized in EEG measurements, not to mention many other more sophisticated functionalities thereof. The outputs of the ECG amplifiers are tunneled into a computer system wherein the amplified analogue signal is digitalized and analysed. The rather complicated system is depicted in the FIG. 1 of the publication.

As more complex EEG measurements require using e.g., 19-25 channels instead of a typical maximum of 3-12 channels supported by an average ECG apparatus, the publication suggests multiplexing a greater number of EEG signals into a lesser number of ECG channels as controlled by the computer system. The disclosed solution is not intended for emergency medicine, but for neurological investigations with multi-channel EEG of patients who are in a stable condition. The publication further describes the statistical computer analysis (Z transform) of the multichannel EEG signal acquired with the multichannel computer-amplifier configuration. The invention does not suit long-term monitoring and could not be used without a special computer and significant amount of digital processing hardware.

As explained above, the poor availability of dedicated FEE monitoring devices is a real shortage causing multiple risks to the emergency medical diagnostics and treatment. Brain disorders should be diagnosed without any unnecessary delay to be able to start optimal treatment right from the very beginning. Even if the expensive EEG monitoring devices were made available at each emergency unit, the medical personnel in charge would not be capable to use of complicated new apparatuses and their features for carrying out initial diagnosis and for starting necessary instant treatments and actions. Devices like the one presented by U.S. Pat. No. 5,287,959 do not solve the usability or even the cost issues either, as they require designing a parallel infrastructure around them for offering the same overall value the current ECG devices are capable of providing.

### SUMMARY OF THE INVENTION

The objective of the present invention is to alleviate the defects found in the EEG recording and monitoring readiness of current primary, short delay/fast response medical care and emergency units. The object is achieved with a solution providing a method and a related apparatus for adapting the measured BEG signals to the characteristic range of the ECG signals according to a number of predetermined physical) factors. Accordingly, the already widespread and routinely used ECG devices as well as the existing ECG infrascture technology can be exploited in measuring the EEG and executing associated further health care actions.

In an aspect of the invention, a method for adapting an electroencephalography (BEG) measurement signal to the characteristic range of an electrocardiography (ECG) measurement signal, is characterized in that said method comprises the steps of
providing a conversion apparatus comprising an input interface for at least functionally connecting with a plurality of electrodes, a signal amplification means, and an output interface,
receiving the EEG measurement signal in the conversion apparatus via said input interface,
processing the received EEG measurement signal by at least said signal amplification means so as to represent the signal, in relation to at least one predetermined parameter, using a parameter value range characteristic to an COG measurement signal, and
transmitting said processed EEG signal through the output interface in order to enable a receiving device to treat said processed EEG signal like an BCG measurement signal.

In the above the term “characteristic range” of an COG measurement signal refers to one or more commonly adopted parameter value ranges, i.e. used industry or de-facto standards, according to which the ECG measuring instruments (˜ECG devices) and features thereof (components, display, etc) have been typically calibrated in relation to the ECG signal input from the electrodes. Such parameters may include signal amplitude that is thus amplified from the lower EEG level to a higher ECG level. The characteristic range may also be interpreted so as to implicitly maintain the readability of the EEG trace even when depicted at the output (display, plotter, etc) of the ECG device as, at least to a predetermined extent, the time-amplitude relationship (˜geometry) of the output trace shall match with the signal representation the medical personnel and other experts are accustomed to see and inspect by means of such equipment. In case of an EEG measurement signal the amplitude of which is typically within 5-250 μV, the gain factor applied by the conversion apparatus could be 20, for example. The gain factor may be made dependent on the properties of the input signal as to be described hereinafter.

Considering other parameters, as the frequency range can be somewhat wider in the ECG than in the EEG, the conversion apparatus shall optionally pre-filter the BEG measurement signal according to the typical BEG monitoring frequency range, so that the destination EC) device or some other device adapted to receive ECG measurement signals, while still utilizing the wider input frequency range, does not receive the signal portion originally existing in the conversion apparatus input signal below or above the typical EEG monitoring frequency range but within the lower and upper limits of the typical ECG monitoring frequency range. Inputting such intermediary frequencies, although being processable by the ECG device, would add noise rather than useful information, and would thus only confuse the device operator or a corresponding person analysing the ECG through the ECG device.

The verb “treat” refers to the actions the receiving (˜destination) device is initially adapted to perform on the ECG signal, i.e. signal reception and processing, for example. The conversion apparatus may be transparent from the viewpoint of the destination device, or it may add new, controllable functionalities thereto as to be described later.

In another aspect, an apparatus for adapting an electroencephalography (EEG) measurement signal to the characteristic range of an electrocardiography (ECG) measurement signal, is characterized in that it comprises
an input interface for receiving an EEG measurement signal captured by a plurality of electrodes,
an input stage functionally connected, in series, with said input interface,
a signal processing means for representing the BEG measurement signal, in relation to at least one predetermined parameter, using a parameter value range characteristic to an ECG measurement signal, and
an output interface for transmitting the processed EEG signal to a receiving device.

Functional entities of the conversion apparatus such as the input stage and signal processing, e.g. amplifiation, means may in practise be merged or further divided into one or more physical elements that execute the associated functionalities.

The input interface provides physical connection, e.g. connectors, to the electrodes or leads connecting to the electrodes. The electrodes may be external to the device or integrated in it forming an aggregate electrode-transformer entity. The input stage adjacent to the input interface typically comprises one or more differential (instrumentation) amplifiers or other means suitable for reducing the common mode to noise possibly present in the EEG measurement signal. Alternatively) attenuation of the common mode noise may be completely entrusted to the receiving device.

The output interface comprises a number of connectors to interface the conversion apparatus with the ECG device. From a technical point of view, the output interface could simply be unipolar, but as the most ECG devices comprise differential input, the output shall often include three connectors to conveniently interface with the destination ECG device's each input electrode lead without need to use additional adapters. Alternatively, the output interface may incorporate the (optionally fixed) leads that are connectable to the inputs of the receiving device. Yet in another alternative, the output interface of the apparatus comprises connectors adapted to directly accommodate or enter the counterpart in the receiving device, i.e. male vs. female connectors. The latter appears particularly attractive option whenever the apparatus is substantially implemented as or included in a module that is connected to the receiving device. The counterpart interface/connector of the receiving destination device may be either internal (within the housing) or external (outer surface), which partly defines the size, casing and voltage supply requirements for the design of the module.

Aforementioned and optional, yet to be disclosed, features of the method and the apparatus according to the invention are further analysed in the detailed description.

The utility of the invention arises from a plurality of issues. First, the provided apparatus can be implemented as a small-sized, one-piece “black box” type device that is light, durable (e.g. physical/electric shock resistant), and structurally relatively simple. Such features imply good overall manageability of the apparatus and trouble-free connectivity to the patient and different cables or connectors at the input/output thereof. Alternatively, the apparatus can be implemented as a module connectable to an ECG device after necessary modifications or via an already-existing interface such as an expansion slot. The price per unit can also be kept low compared to the prices of independent EEG instruments. This fact enables manufacturing the apparatuses even as disposable units. Only one EEG channel is necessary for simple diagnostic use, whereas more channels can be implemented in the devices targeted to more demanding analysis. The existing ECG infrastructure including the relating hardware, (wireless) data transfer features and intellectual know-how can be now exploited in the context of EEG respectively. The device is easy to use, i.e. the paramedics and other medical personnel may only take a crash course and start operating it. In the simplest form, the standard ECO electrode leads connected to the input of the ECG device are also directly connectable to the output of the conversion apparatus, whereas the EEG electrodes connected to the input of the apparatus are removably attached to the scalp (or forehead skin, earlobes, etc.) of the patient. No fine-tuning parameters or twiddling with various adapters is advantageously required. A number of adjustment means (e.g. is buttons, switches, computer interface, (touch-sensitive) display, etc) may be offered for apparatus control purposes, but they shall be optional features.

The use of the apparatus as planned with emergency units, health centers, etc equipped with ECG enables determining rapid EEG-based diagnosis and carrying out required medical interventions accordingly and without delay in various emergency scenarios previously occurring unduly far from dedicated EEG equipment. This is likely to alleviate the consequences of acute brain disorders and trauma, and even save patients, lives. Naturally the invention is correspondingly applicable in environments not primarily intended for emergency care and lacking the dedicated EEG devices, such as hospital bed departments.

In one embodiment of the invention, the invention is utilized in an emergency scenario wherein a patient suffering from a potential brain electrical disorder is picked up by paramedics and the device of the invention is exploited to enable immediate diagnostics so that the initial treatment can be started without a delay. The measured EEG is transferred via a wireless transceiver to a remote location, e.g. intensive care unit, for enabling expert analysis and for obtaining instructions concerning (immediate) medication or other preparatory actions.

Dependent claims disclose embodiments of the invention.

### BRIEF DESCRIPTION OF THE DRAWINGS

Hereinafter the invention is described in more detail by reference to the attached drawings, wherein

FIG. 1 depicts the overall scenario of said one embodiment of the invention.

FIG. 2 is a block diagram of an electronic apparatus according to said one embodiment of the invention.

FIG. 3 is a flow diagram representing the potential steps of the method of the invention.

FIG. 4 is a trace of human BEG captured simultaneously via both a dedicated EEG device and an BCG device connected to the conversion apparatus of the invention.

FIG. 5 depicts a module concept in which the apparatus of the invention is implemented as a module connectable to an ECG device.

### DETAILED DESCRIPTION OF THE EMBODIMENTS OF THE INVENTION

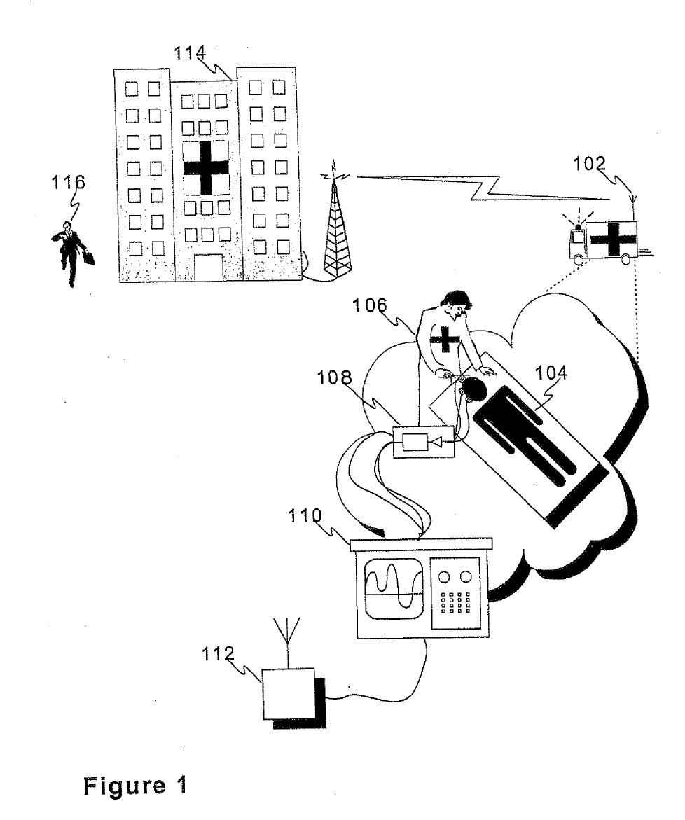

FIG. 1 visualizes a fictive operating situation of the conversion apparatus by way of example only. An ambulance 102 has reached an accident site and picked up a patient 104 with altered consciousness. A paramedic 106 is busy in conducting a diagnosis and giving emergency medical treatment.

The apparatus of the invention 108 receives BEG measurement signals from e.g. three EEG electrodes that are positioned on hairless areas of the patient's head/scalp, such as the frontal forehead or mastoids, or on hairy locations such as the vertex. The apparatus 108 outputs the BEG signal as better adapted to the COG measurement signal range so that the ECG device 110 may process it like an ECG measurement signal and represent it to the paramedic 106 via a display or a plotter, for example. Further, in the visualized example the ECG output signal or a number of predetermined parameters derived therefrom are preferably wirelessly transmitted forward via a radio transmitter or transceiver 112 to the destination hospital 114, wherein medical personnel, e.g. specialists, may analyse it, provide more specific treatment instructions to the paramedic 106, and prepare to execute optimum procedures when the patient 104 arrives. Based on the received information, also additional personnel 116 can be called in.

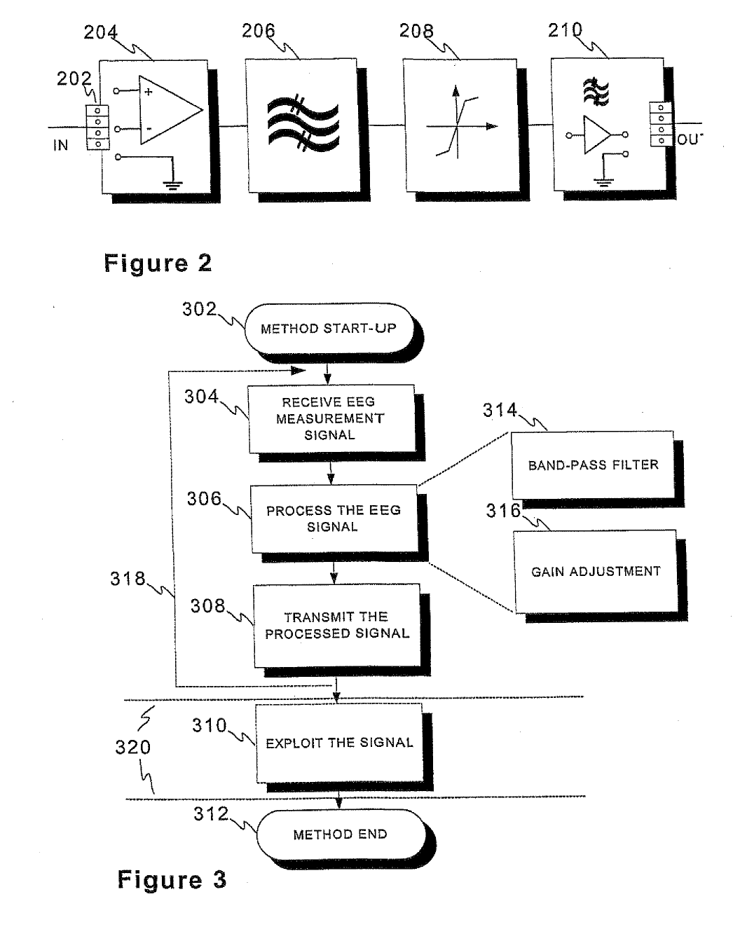

FIG. 2 discloses a block diagram of one possible embodiment of the apparatus 108. It should be noted that the depicted blocks represent essentially functional entities, which enables a person skilled in the art to further divide them into even smaller sub-blocks or conversely, to combine them to form higher level aggregate entities in view of the initial configuration shown in the figure. For example, gain block 208 and input stage 204 may be merged together.

Block 202 refers to the mechanical/physical input interface for receiving the EEG measurement signal as captured by the electrodes. One or more electrodes can be either integrated in the apparatus housing in which case such interface comprises the electrode(s) as well (or conceptually vice versa, i.e. the apparatus is integrated in the electrodes), or the interface comprises merely connectors for attaching to the electrodes (or in most cases, the EEG electrode leads). Further, the final number of electrodes or electrode connectors, e.g. three, in the interface 202 depends on the preferred number of channels the apparatus is configured to simultaneously receive.

Block 204 refers to an input stage that shall optionally enable EEG recordings with an appropriate signal-to-noise ratio even when the electrical coupling across the electrode-skin interface is not optimal. It thus comprises one or more, preferably differential (instrumentation), amplifiers co-operating with the physical interface 202. Differential input stages are generally advantageous for rejecting common-mode noise induced in the bioelectric measurement signals such as the EEG measurement signal entering the apparatus 108 via the input interface 202. Technical features of the input stage 204 shall preferably incorporate high input impedance and high CMRR (Common-mode rejection ratio) through the measuring range. E.g. 50 or 60 Hz hum radiated by various near-by power cables to the signal inputs is, in the scenario of the current invention, particularly harmful as it occurs close to the monitored frequency range. Therefore the differential amplifiers shall preferably have a relatively high CMRR of order 100 000, i.e. 100 decibels, for example. Inputs are typically capacitively (AC) coupled so as to lower the stability requirements set for the electrode attachment, but also DC coupled inputs may be used. In addition, other properties or functions such as overvoltage protection, fault-protection circuitry for patient safety, measurement of the electrode impedance, etc can be optionally implemented to the blocks 202 and 204.

Established patient safety provisions concerning, for example, a scenario of a single failure may, in minimum case, be attained by capacitive separation in the AC coupled case or by series-connected resistors in the DC coupled case. Another benefit offered by the current invention is that galvanic decoupling does not need to be implemented as part of the apparatus 108. This is because the ECG device provides decoupling, if the output signal of the apparatus 108 is received by an ECG device that utilizes galvanic signal isolation and if the apparatus 108 is left floating with respect to the ground level. This can be implemented either by battery-driving (using either rechargeable or disposable batteries) the apparatus 108 or by providing isolated supply voltage thereto from e.g. the ECG device. Naturally also conventional protection against static discharges (from the medical personnel hands etc) shall be applied.

Block 206 refers to frequency range adjustment procedures as mentioned herein earlier. In a typical embodiment the frequency range is limited with filters to a bandwidth that provides a sufficient amount of information for diagnostic purposes, for instance from 2 to 30, 40, or 50 Hz. Even simple RC filters may be used, although active filters with steeper roll-off give better results, Thus e.g. a Butterworth filter, a Bessel filter, a Chebyshev filter and various other filter forms are applicable depending on the design requirements. Although the apparatus 108 can be implemented via analogue electronics, also digital implementation employing e.g. digital signal processors for filtering and/or other functions is possible.

Block 208 visualizes a non-linear gain feature comprising e.g. one or more operational amplifiers with a nonlinear feedback circuit. Introducing a predetermined amount of non-linearity to the amplification procedure may be advantageous on account of the considerable dynamic range utilized and possible high-amplitude noise transients in the received EEG measurement signal, which might otherwise cause saturation of the apparatus 108 or of the ECG device. Nevertheless, a person skilled in the art shall implement the gain as he wishes, and the non-linearity aspect, when present (notice the sketch of a gain input-output curve in block 208), may be either fixed, i.e. the overall gain factor is e.g. 20 until die EEG measurement signal level ±200 μV is reached beyond which the gain factor is reduced, e.g. to two, or alternatively, the operator of the apparatus 108 may be provided with an opportunity to adjust the gain functionality via available UI (user interface) means such as knobs, switches, buttons, or a more sophisticated control interface. In the “black box” type embodiment the settings are fixed and the apparatus 108 is preferably ready for use out-of-the-box.

Block 210 depicts an output interface of the apparatus 108 for connecting to an ECG device. Although a straightforward implementation of the apparatus 108 would provide a unipolar output, the output block 210 preferably comprises connectors for directly interfacing the commonly used snap-on fasteners or other type connectors of the (differential) input signal leads of the ECG device. The connectors as well as the electrodes and cables are farther advantageously color-coded in accordance with the standard practice in the field. Optionally block 210 also includes output gain unit and/or a band-pass filter.

The housing of the apparatus 108, advantageously being rater small (˜matchbox or even coin size, e.g. 3 cm×5 cm×1.5 cm, or less) and light (tens or mostly hundreds of grams), may be attached to the patient (head, arm, body, etc) or any near-by surface by utilizing e.g. velcro so as to avoid disturbing the ongoing diagnostic measures or treatment.

FIG. 3 discloses one example of a method for carrying out the inventive concept by the apparatus 108. In method start-up 302 miscellaneous preparatory actions are taken to enable the execution of the subsequent method steps. For example, the conversion apparatus 108 is obtained, and the necessary signal provision means such as leads (˜cables) are connected to the patient 104 with electrodes (EEG electrode leads), the apparatus 108 (other end of the EEG electrode leads to the input interface, and output lead(s), i.e. ECG measurement signal leads, towards the ECG device to the output interface), and the ECG device 110 (other end of the BCG measurement signal leads to the input). The necessary devices such as the apparatus 108 and BCG device 110 shall also be turned on. Alternatively, the apparatus 108 shall power-up automatically in response to a predetermined event that is detected. Such events may include plugging in one or more leads, for example. In the optional case of adjustable internal parameters the operator of the apparatus 108 may either change them or just verify the current settings, and generally test the functioning of the device. Further, a connection between the ECG device 110 and a remote receiver via e.g. a locally available radio transceiver can already be established at this stage. This applies especially for continuous measurement data transmission between the ECG device 110/transceiver 112 and the remote receiver, whereas in case the measurement data to be transferred only relate to a predetermined period, data can be first gathered to the ECG device 110 and then forwarded to the transceiver 112 in its entirety during a separate method step (not visualized). Transmission format for the ECG/EEG data shall be selected so as to flawlessly interface with the data reception capabilities of the remote receiver. Often in telemedicine e.g. different fax formats (the actual resolution being defined by e.g. (ITU-T) Groups 1-4 specifications and transmission rates by V.27-V.34bis standards) are used.

Step 304 refers to receiving the EEG measurement signal in the apparatus 108 via the input interface and input stage thereof. Electrical activity created by the patient's brain is initially captured by a number of electrodes located on the patient's head (scalp). The measurement signal is then conveyed by the connecting leads to the input interface. The input stage implemented by e.g. differential amplifiers introduces simple pre-processing to the input EEG measurement signal by amplifying it and diminishing the common-mode disturbance signals possibly present therein.

Step 306 indicates the actual processing of the received EEC measurement signal within the apparatus 108 in relation to one or more predetermined parameters such as signal amplitude, magnitude, frequency, etc. Processing may thus indicate e.g. gain adjustment 316 (amplification) of the received EEG signal to the characteristic value range of the typical ECG measurement signals. Further, such processing step may refer to frequency domain related actions like signal (band-pass) filtering 314 as described hereinbefore. The execution order of the signal filtering 314 and gain adjustment 316 steps may also be reversed with particular reference to the description of FIG. 2, wherein the functional blocks of the apparatus 108 and various alternatives for their implementation were adduced.

Step 308 includes transmission of the processed BEG measurement signal through the output interface of the apparatus 108. The ECG device 110 (or some other device adapted to receive EEG measurement signal) functionally connected to the interface shall then receive the processed EEC measurement signal and consider it as a standard ECG measurement signal captured by the ECG electrodes.

Step 310 that is separated from adjacent actions with dotted lines 320 for clarity reasons denotes actions taking place outside the apparatus 108. The existing ECG infrastructure, e.g. features of the ECG devices, can now be exploited, which anticipates additional synergy benefits. The processed BEG signal is received by another device such as the ECG device 110 that may optionally further process and adapt the signal and transmit it forward either wirelessly or by wire, store it, show the trace or other information derived from the received data on an external or internal display, etc. The derived information may include a number of indexes describing the brain activity, e.g. medicinal actions or anaesthesia depth. The constructed aggregate system thus comprises the apparatus 108 of the invention and selected parts of the existing ECG infrastructure such as the ECG devices, data transmission facilities, analysis, display and storage means, etc.

The apparatus 108 may simultaneously adapt a plurality of channels instead of a single one, if provided with a sufficient number of input/output connectors and necessary internal electronics, as being clear to a skilled person. The channels may have independent differential inputs, or they may share a common reference like in many dedicated EEG devices.

In step 312 the method execution is ended in the apparatus 108. Dotted loop 318 visualizes the continuous nature of the process, i.e. the apparatus 108 substantially functions in a real-time fashion until the measurement procedure is finished. Alternatively, the apparatus 108 includes memory to store the EEG signal, whereby the processed) EEG signal can be transferred to the receiving device at a later time in response to a triggering procedure such as pressing a button or receipt of a control signal if provided with a suitable receiver/interface.

In addition to analogue circuits, the invention may be implemented through digital electronics, i.e. digital circuits such as digital logic chips, microprocessors, microcontrollers, digital signal processors, etc, However, the electrode (lead) output signal is typically analogue and thus the apparatus 108, although being substantially digital, should still include at least an A/D converter, i.e. analogue components. In case electrodes with integrated A/D converters are used but not included in the apparatus 108, analogue electronics may be completely omitted provided the output is also digital. Insofar as the apparatus 108 is at least partially implemented via (re)programmable digital means, the code for the execution of the proposed method can be stored and delivered on a carrier medium like a floppy, a CD, a hard drive or a memory card.

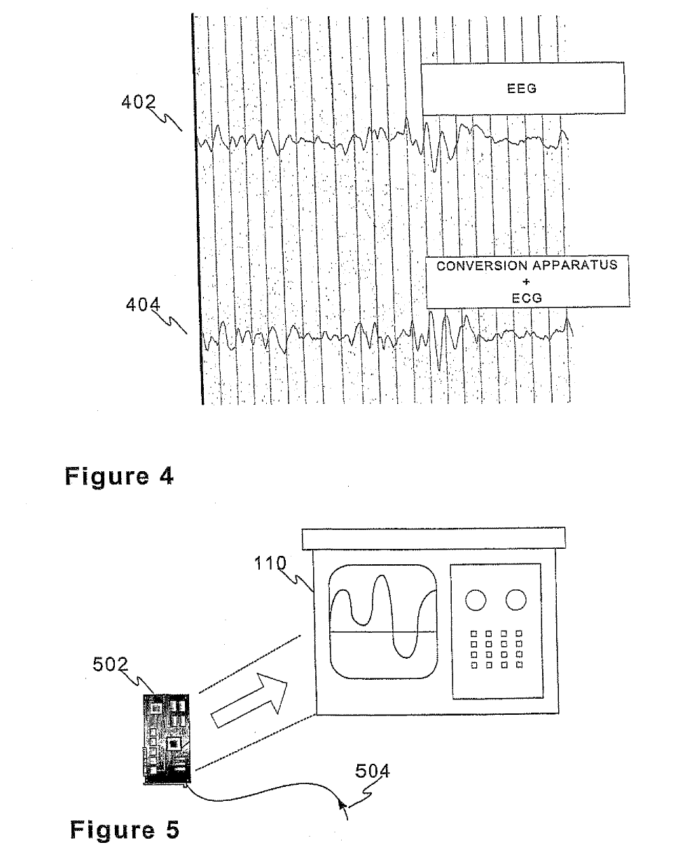

FIG. 4 illustrates specimen traces of a human EEG captured simultaneously via a dedicated EEG device and an ECG device connected to the apparatus of the invention. Upper trace 402 belongs to the dedicated BEG device whereas the lower one 404 corresponds to the arrangement in which a prototype of the apparatus according to the invention receives the EEG measurement signal and adapts it for the ECG device. Diminutive differences between the traces are due to the different signal filtering and gain characteristics applied in the two solutions

FIG. 5 depicts a use case of the invention wherein a module 502 includes the essential functionalities of the apparatus of the invention as described hereinbefore.

The module thus provides the ECG 110 or another receiving device with similar means for adapting the EEG measurement signal including an input interface and preferably differential input stage for receiving the EEG measurement signal 504, a signal processing means, and an output interface for coupling to the destination device. The required functionalities can be implemented by a predetermined hardware configuration (traditional analogue circuit arrangements, ASICs (Application Specific Integrated Circuit), programmable logic, etc) or by combination of more generic hardware (multi-purpose microprocessors/DSPs/microcontrollers) and use-specific software.

The module may be installed in the housing of the receiving device as an internal extension card, or can be encased in a dedicated housing that is connected to the interface on the exterior surface of the device. Instead of electrode leads or lead connectors, the output interface of the module 502 is preferably designed to directly fit the receiving connector of the ECG device 110 such that using any additional adapters is avoided. In that case the ECG should have been designed to support retrofit extensions. Alternatively, the ECG device 110 has to be specifically modified to accommodate the module. The module 502 may be equipped with a control interface through which the functionalities thereof and optionally of the destination device can be controlled, or the ECG device 110 may bear ready-fitted capability for controlling extension products and utilizing their additional features. Certain functionalities of the stand-alone apparatus 108 may be furnished in the module scenario by capitalizing the existing features of the destination device 110. If, for example, signal arriving at the standard (ECG) input of the device 110 can be internally tunnelled into the module 502, the actual electrode leads or lead connectors may be omitted from the input interface thereof, it may suffice to attach the module 502 to the data bus of the device 110. Such tunnelling can be actuated through switchable input (EEG/ECG) that is either retrofitted to the destination device or ready available.

The scope of the invention can be found in the following claims. However, utilized method steps, components, interfaces, etc may depend on a particular use case still converging to the basic ideas presented herein, as appreciated by a skilled reader. For instance, the invention may also be utilized in veterinary medicine, although the above examples were given in the context of human medicine only.

1. A method for adapting an electroencephalography (EEG) measurement signal to the characteristic range of an electrocardiography (ECG) measurement signal, characterized in that said method comprises the steps of
providing a conversion apparatus comprising an input interface for at least functionally connecting with a plurality of electrodes, a signal amplification means, and an output interface,
receiving the EEG measurement signal in the conversion apparatus via said input interface,
processing the received EEG measurement signal by at least said signal amplification means so as to represent the signal, in relation to at least one predetermined parameter, using a parameter value range characteristic to an ECG measurement signal, and
transmitting said processed EEG signal through the output interface in order to enable a receiving device to treat said processed EEG signal like an ECG measurement signal.

2. The method of claim 1, wherein said processing step indicates amplifying, preferably in a non-linear manner, the EEG measurement signal.

3. The method of claim 1, wherein said processing step indicates filtering the EEG measurement signal by a number of filters provided in the conversion apparatus.

4. The method of claim 1, wherein said conversion apparatus further comprises a differential input stage.

5. An apparatus for adapting an electroencephalography (EEG) measurement signal to the characteristic range of an electrocardiography (ECG) measurement signal, characterized in that it comprises
an input interface (202) for receiving an EEG measurement signal captured by a plurality of electrodes,
an input stage (204) functionally connected, in series, with said input interface,
a signal processing means (206, 208) for representing the EEG measurement signal, in relation to at least one predetermined parameter, using a parameter value range characteristic to an ECG measurement signal, and
an output interface (210) for transmitting the processed EEG signal to a receiving device.

6. The apparatus of claim 5, wherein said input stage is differential in order to reduce the common mode noise possibly present in the EEG measurement signal.

7. The apparatus of claim 5, wherein said signal processing means comprises an amplification means (208) to adjust the amplitude of the received EEG measurement signal.

8. The apparatus of claim 7, wherein a gain factor is selected so as to convert a predetermined amplitude range of EEG measurement signals to a predetermined amplitude range of ECG measurement signals.

9. The apparatus of claim 8, wherein the gain provided by said amplification means (208) is non-linear.

10. The apparatus of claim 5, wherein said signal processing means comprises at least one filter (206) for limiting the frequency range of the received EEG measurement signal.

11. The apparatus of claim 10, wherein the pass band of said at least one filter resides within range defined by a lower threshold below 5 Hz and an upper threshold below 50 Hz.

12. The apparatus of claim 10, wherein said filter is selected from the group consisting of: an RC filter, a Butterworth filter, a Bessel filter, and a Chebyshev filter.

13. The apparatus of claim 5, wherein said input interface (202) comprises one or more electrodes.

14. The apparatus of claim 5, wherein said input interface (202) comprises a number of connectors to receive EEG electrodes or the corresponding EEG electrode leads.

15. The apparatus of claim 5, wherein said input interface (202) comprises a plurality of electrode leads.

16. The apparatus of claim 5, wherein said input stage (204) includes a feature selected from the group consisting of: CMRR (common mode rejection ratio) higher than 80 dB, AC coupled inputs, DC coupled inputs, an overvoltage protection, and measurement of the electrode impedance.

17. The apparatus of claim 5, wherein said output interface (210) comprises a number of connectors for receiving ECG signal leads.

18. The apparatus of claim 17, wherein said connectors bear a shape mimicking the shape and size used in the ECG electrodes.

19. The apparatus of claim 5, wherein said output interface (210) is connectable to the ECO signal input interface of said receiving device.

20. The apparatus of claim 5, wherein said output interface (210) comprises a unipolar output.

21. The apparatus of claim 5, wherein said output interface (210) comprises an element selected from the group consisting of: a band-pass filter, and a gain unit.

22. The apparatus of claim 5, being either battery-driven or configured to receive an isolated voltage supply from the ECG device.

23. The apparatus of claim 5, comprising a housing the size of which substantially corresponds to a matchbox or a coin.

24. The apparatus of claim 5, being a disposable apparatus.

25. The apparatus of claim 5, comprising substantially analogue or digital electronics.

26. The apparatus of claim 5, comprising a control interface for receiving user input to adjust a number of operating parameters.

27. An electrode arrangement comprising a number of electrodes for capturing an EEG signal and an apparatus as defined by claim 5.

28. A system comprising a number of devices adapted to receive and process ECG measurement signals and an apparatus as defined by claim 5 to adapt an EEG measurement signal to the characteristic range of an ECG measurement signal.

29. The system of claim 28, adapted to determine a number of indexes from the adapted EEG signal, said indexes representing brain function.

30. The system of claim 28, further comprising a transmitter adapted to transmit the received, adapted EEG measurement signal or information derived therefrom to a remote party.

31. The system of claim 30, wherein said transmitter is a wireless transmitter.

32. Use of an apparatus as defined by claim 5 for conducting EEG measurements by an ECG device.

33. The use as defined by claim 32, taking place either in emergency medicine or long-term monitoring.

34. A module connectable to an electronic device capable of receiving and processing ECG signals, said module comprising the apparatus as defined by claim 5.

35. The module of claim 34, being an extension card to be installed in said electronic device.

36. The module of claim 34, comprising a housing provided with said output interface for coupling to said electronic device.
